(19) United States
(12) Patent Application Publication
(10) Pub. No.: US 2021/0285909 A1
(43) Pub. Date: Sep. 16, 2021

(54) METHOD FOR MEASURING CONCENTRATION OF BIOMETRIC MEASUREMENT OBJECT BY USING ARTIFICIAL INTELLIGENCE DEEP LEARNING

(71) Applicant: I-SENS, INC., Seoul (KR)

(72) Inventors: Seok-Won LEE, Gyeonggi-do (KR); Byeong Keun KANG, Seoul (KR); Myeongho LEE, Seoul (KR); Hakhyun NAM, Seoul (KR); Hyeonseok LEE, Busan (KR)

(73) Assignee: I-SENS, INC., Seoul (KR)

(21) Appl. No.: 17/257,545

(22) PCT Filed: Jul. 13, 2018
(86) PCT No.: PCT/KR2018/007934

§ 371 Date: Dec. 31, 2020

(30) Foreign Application Priority Data
Jul. 11, 2018 (KR) 10-2018-0080372

(51) Int. Cl. G01N 27/327; G06K 9/00; G06K 9/62; G06N 3/08

(52) U.S. Cl. CPC G01N 27/3274; G06N 3/08; G06K 9/6265; G06K 9/00147

(57) ABSTRACT

The present invention has been made in an effort to provide an analyte concentration measurement method using artificial intelligence deep learning, capable of extracting useful features that are not known in advance by humans through deep learning using artificial neural networks by imaging input signals obtained during the measurement time from samples with information (labels) to construct a data set and estimating a result value by applying an algorithm obtained through learning in this way, compared with conventional measurement techniques that are applied by devising formulas or methods that directly extract features for a long time by experts in related fields in order to extract effective features.

### TECHNICAL FIELD

The present invention relates to an analyte concentration measurement method using artificial intelligence deep learning, and more particularly, to an analyte concentration measurement method using artificial intelligence deep learning, which recognizes an area corresponding to an analyte by converting a signal obtained from a sensor by applying deep learning-based artificial neural network techniques, and predicts a most suitable type and concentration of the analyte by extracting each element necessary for determining type or concentration of the analyte in the area.

### BACKGROUND ART

In general, one of the most important criteria for in-vitro diagnosis products is the accuracy of the measurement results.

In such products, when an analyte is present, a detection sensor outputs a signal, and an analyzer recognizes it and applies a predetermined calibration curve or algorithm to send quantitative or qualitative results.

The accuracy of the measurement result may be influenced by an interference action caused by various variables such as an interferent, an external environment, and sample properties.

In the case of an electrochemical sensor utilized for blood glucose measurement, an output electrical signal may be influenced due to a change in a diffusion coefficient toward an electrode or a reaction rate at an electrode surface due to a change in blood properties such as presence of a material other than an analyte oxidized at the electrode surface or viscosity.

In order to minimize or eliminate this effect, a method of estimating and reflecting a degree of interference of a measured object by sending various types of input signals to the sensor has been used.

For example, as disclosed in Korean Patent Registration No. 1666978, in a method for measuring concentration of an analyte in a biological sample, a redox enzyme and an electron transfer medium that can catalyze a redox reaction of the analyte were fixed, a liquid biological sample was injected into a sample cell equipped with a working electrode and an auxiliary electrode, a first sensitive current was obtained at a feature point of at least one point in time by initiating the redox reaction of the analyte and applying a constant DC voltage to the working electrode such that an electron transfer reaction can proceed, a second sensitive current was obtained at least two times or more by applying a Λ-stepladder-type perturbation potential after a constant DC voltage was applied, a predetermined feature was calculated from the first sensitive current or the second sensitive current, and the concentration of the analyte was calculated by using a calibration formula consisting of at least one feature function to minimize the influence of at least one interfering substance in the biological sample, so as to measure the concentration of the analyte in the biological sample.

That is, the degree of interference was estimated by using a mathematical method such as Multiple Linear Regression using parameter values derived from a predetermined signal, and the measured value was determined.

A drawback of this method is to output an unexpected and inaccurate result when it cannot distinguish or detect a given rule of previously extracted features or input command.

Although most features are focused on excluding an influence of hematocrit, when at least two interfering species other than hematocrit are combined in combination to affect the concentration of the bioanalyte, the features and a calibration equation using these features have to be corrected, but otherwise, it may cause fatal problems.

In addition to the hematocrit, even when at least two interfering species are combined to affect the concentration of the bioanalyte, there was a problem in that a biometric strip was abused because the abnormality was not known.

Furthermore, for external environmental factors, for example, a value of the temperature feature is a value obtained through a temperature sensor attached to a meter, and when there is a sudden change in the surrounding environment, it is difficult to measure the correct temperature immediately and there are many cases where time is required to achieve temperature equilibrium.

Therefore, most meters require a user to wait a certain amount of time to equilibrate the temperature in order to use the meter when the user is in a suddenly changed environment (ex. enters a warm house from outside on a cold winter day).

### DISCLOSURE

The present invention has been made in an effort to provide an analyte concentration measurement method using artificial intelligence deep learning, capable of extracting useful features that are not known in advance by humans through deep learning using artificial neural networks by databasing input signals obtained during the measurement time from samples with information (labels) and estimating a result value by applying an algorithm obtained through learning in this way, compared with conventional measurement techniques that are applied by devising formulas or methods that directly extract features for a long time by experts in related fields in order to extract effective features.

The present invention has been made in an effort to provide an analyte concentration measurement method using artificial intelligence deep learning to obtain features that may be classifiers for classifying groups without the need to search for a separate feature each time, compared with conventional measurement techniques that can be a regression model that estimates a specific value, which takes a lot of time and effort to find a feature each time at which a shape of an input waveform entering a sensor including a sample changes.

The present invention has been made in an effort to provide an analyte concentration measurement method using artificial intelligence deep learning, capable of automatically calibrating features and calibration formulas using these features when at least two interfering species other than hematocrit are combined in combination to affect the concentration of the bioanalyte.

The present invention has been made in an effort to provide an analyte concentration measurement method using artificial intelligence deep learning, capable of announcing special abnormalities when at least two interfering species other than hematocrit are combined in combination to affect the concentration of the bioanalyte.

In addition, the present invention has been made in an effort to provide an analyte concentration measurement method using artificial intelligence deep learning, capable of providing constant accuracy even when measuring the concentration of a bioanalyte without waiting for a certain period of time for stabilization of changes in an external environment against sudden changes in the external environment.

An exemplary embodiment of the present invention provides a method for measuring concentration of an analyte material in a biological sample, using artificial intelligence, including: injecting a liquid biological sample into a sample cell having a working electrode and an auxiliary electrode, in which an electron transport medium and a redox enzyme which is capable of catalyzing a redox reaction of an analyte are fixed; obtaining a first sensitive current at a characteristic point of at least one point of time by applying a constant DC voltage to the working electrode to initiate the redox reaction of the analyte and to proceed with an electron transfer reaction; obtaining a second sensitive current at two or more points of time by applying a Λ-stepladder-type perturbation potential after applying the constant DC voltage; calculating a predetermined feature from the first sensitive current or the second sensitive current; and correcting concentration of the analyte by using an calibration formula composed of at least one feature function by artificial intelligence learning such that an influence of at least one interfering substance in the biological sample is minimized.

In accordance with an analyte concentration measurement method utilizing artificial intelligence deep learning according to an exemplary embodiment of the present invention, an object of the present invention capable of extracting useful features that are not known in advance by humans through deep learning using artificial neural networks by databasing input signals obtained during the measurement time from samples with information (labels) and estimating a result value by applying an algorithm obtained through learning in this way, compared with conventional measurement techniques that are applied by devising formulas or methods that directly extract features for a long time by experts in related fields in order to extract effective features, is to obtain features that may serve as a classifier for classifying a group without the need to search for a separate feature each time, compared with conventional measurement techniques, which were to devise and apply a formula or method that directly extracts features for a long time by experts in related fields in order to extract effective features, can be a regression model that estimates a specific value, which takes a lot of time and effort to find a feature each time at which a shape of an input waveform entering the sensor containing the sample changes.

In addition, in accordance with an analyte concentration measurement method utilizing artificial intelligence deep learning according to an exemplary embodiment of the present invention, it is possible to automatically correct features and calibration formulas using these features when at least two interfering species other than hematocrit are combined in combination to affect the concentration of the bioanalyte.

In accordance with an analyte concentration measurement method utilizing artificial intelligence deep learning according to an exemplary embodiment of the present invention, it is possible to announce special abnormalities when at least two interfering species other than hematocrit are combined in combination to affect the concentration of the bioanalyte.

In accordance with an analyte concentration measurement method utilizing artificial intelligence deep learning according to an exemplary embodiment of the present invention, it is possible to provide constant accuracy even when measuring the concentration of a bioanalyte without waiting for a certain period of time for stabilization of changes in an external environment against sudden changes in the external environment.

### DESCRIPTION OF THE DRAWINGS

FIG. 1 illustrates an internal configuration diagram of a blood glucose measurement device using artificial intelligence deep learning according to an exemplary embodiment of the present invention.

FIG. 2 illustrates a flowchart of a blood glucose measurement method using artificial intelligence deep learning according to an exemplary embodiment of the present invention.

FIG. 3 illustrates a detailed view of an artificial intelligence learning calibrator in an internal configuration diagram of a blood glucose measurement device using artificial intelligence deep learning according to an exemplary embodiment of the present invention.

FIG. 4 illustrates a graph showing a Λ-stepladder-type perturbation potential used in a blood glucose measurement method using artificial intelligence deep learning and a corresponding sensitive current according to an exemplary embodiment of the present invention.

FIG. 5 illustrates an exemplary diagram showing results of a regression model in which blood glucose estimation is performed depending on a blood glucose estimating method using a conventional multiple regression method and an analyte concentration measurement method utilizing a deep learning method using an artificial neural network according to an exemplary embodiment of the present invention.

FIG. 6 illustrates a graph showing robustness of an algorithm according to noise when a random noise of a certain magnitude is applied to an input signal depending on an analyte concentration measurement method utilizing artificial intelligence deep learning according to an exemplary embodiment of the present invention.

FIG. 7 illustrates an exemplary diagram showing results of a regression model in which blood glucose estimation is performed when a temperature feature is not included and when the temperature feature is included depending on an analyte concentration measurement method utilizing artificial intelligence deep learning according to an exemplary embodiment of the present invention.

FIG. 8A and FIG. 8B respectively illustrate graphs showing robustness of an algorithm when an ambient temperature is higher than a room temperature and lower than the room temperature depending on an analyte concentration measurement method utilizing artificial intelligence deep learning according to an exemplary embodiment of the present invention.

### MODE FOR INVENTION

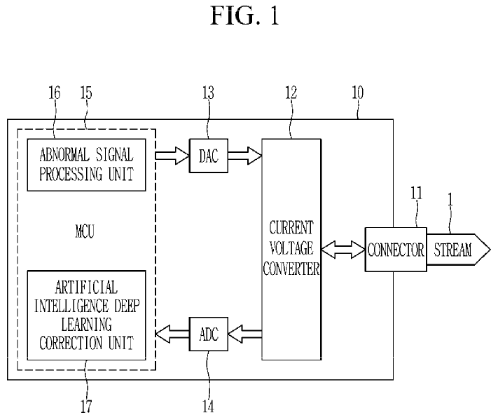

Hereinafter, an analyte concentration measurement method utilizing a deep learning method using an artificial neural network according to an exemplary embodiment of the present invention will be described in detail with reference to FIG. 1 to FIG. 6.

In the present specification, the analyte concentration measurement method utilizing the deep learning method using the artificial neural network according to an exemplary embodiment of the present invention is only an example for describing the present invention, and the scope of the present invention is not limited by the exemplary embodiment.

Hereinafter, a blood glucose measurement method utilizing artificial intelligence deep learning according to an exemplary embodiment of the present invention will be described with reference to FIG. 1 to FIG. 4.

FIG. 1 illustrates an internal configuration diagram of a blood glucose measurement device using artificial intelligence deep learning according to an exemplary embodiment of the present invention.

As illustrated in FIG. 1, the blood glucose measurement device 10 utilizing artificial intelligence deep learning according to an exemplary embodiment of the present invention may provide blood glucose measurement values depending on an optimized artificial intelligence-based deep learning algorithm for blood glucose estimation capable of improving algorithm accuracy, precision, and interference correction performance including hematocrit beyond a conventional multiple linear regression method according to a blood glucose measurement method using the artificial intelligence deep learning by applying a Λ-stepladder-type perturbation potential after applying a certain voltage while maintaining a structure of an existing electrochemical biosensor, i.e., a pair of working electrodes and auxiliary electrodes of a strip 1.

The blood glucose measurement device 10 using artificial intelligence deep learning according to an exemplary embodiment of the present invention may also efficiently calculate an artificial intelligence-based deep learning algorithm for estimating blood glucose that is optimized even in limited hardware, so that the calculation time may be within 8 s.

The blood glucose measurement device 10 using artificial intelligence deep learning according to an exemplary embodiment of the present invention may be configured such that, when the electrochemical biosensor strip 1 is mounted on a connector 11, the connector 11 is electrically connected to a current-voltage converter 12, and a microcontroller (MCU) 15 may apply a constant voltage through a digital-analog converter circuit (DAC) 13 and a Λ-stepladder-type perturbation potential to the working electrode of the strip 1. To this end, firmware of the blood glucose measurement device 10 using artificial intelligence deep learning according to an exemplary embodiment of the present invention first stores a constant capable of generating a predetermined triangular wave circulating voltage in a memory, records a predetermined constant to the register of the DAC 13 when applying a constant voltage, and increases or decreases the constant stored in the memory at a predetermined period of time to record it in a register of the DAC 13 when applying the Λ-stepladder-type perturbation potential. However, the waveform of the Λ-stepladder-type perturbation potential exemplified herein is merely an example, but the present invention is not limited to this example, and includes all waveforms of a circulating voltage that are natural to the workers in the project.

The microcontroller 15 applies the corresponding voltage between two electrodes of the strip depending on the constant recorded in the register of the DAC 13.

When the constant voltage is applied and the Λ-stepladder-type perturbation potential is applied, a response current measured through the strip 1 may be measured directly by an analog-digital converter circuit (ADC) 14 through the connector 11 and the current-voltage converter 12.

On the other hand, the blood glucose measurement device 10 using artificial intelligence deep learning according to an exemplary embodiment of the present invention further includes an abnormal signal processing unit 16 and an artificial intelligence deep learning algorithm calculation unit 17, and when the strip 1 is poorly connected to the connector 11 or an abnormal signal due to abnormal blood injection or hardware is detected, the abnormal signal processing unit 16 may notify this through an alarm, a display, etc., and may prevent the artificial intelligence deep learning correction unit 17 from performing unnecessary calculations.

The artificial intelligence deep learning algorithm calculation unit 17 may obtain a blood glucose measurement value from a response current measured through the strip 1 by an optimized artificial intelligence deep learning blood glucose measurement algorithm within 8 s.

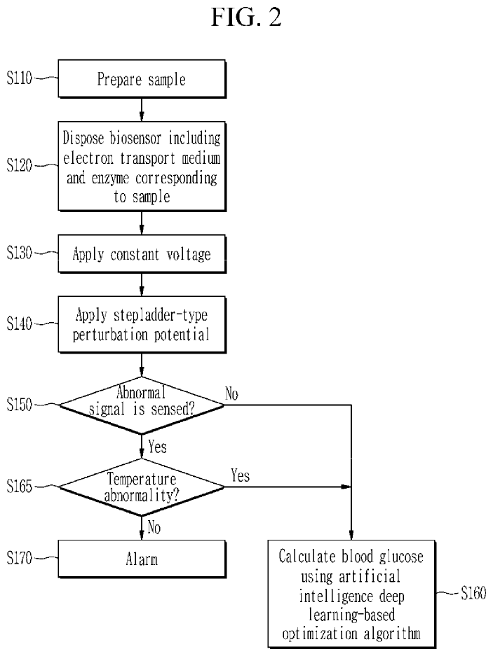

Hereinafter, a blood glucose measurement method utilizing artificial intelligence deep learning according to an exemplary embodiment of the present invention will be described with reference to FIG. 2.

FIG. 2 illustrates a flowchart of a blood glucose measurement method using artificial intelligence deep learning according to an exemplary embodiment of the present invention.

Referring to FIG. 2, the blood glucose measurement method the using artificial intelligence deep learning according to the exemplary embodiment of the present invention includes: preparing a sample (S110); loading, e.g., an electrochemical face-to-face biosensor strip 1 in a biometric information measurement device 10 (S120), applying a constant voltage through a current-voltage converter 12 at a moment when blood soaks a working electrode and an auxiliary electrode of the electrochemical biosensor 1, as the biosensor strip 1 contacts the sample (blood); and continuously applying a Λ-stepladder-type perturbation potential at an end of an applied voltage after the constant voltage is applied (S140).

The abnormal signal processing unit 16 determines whether a sensitive current is an abnormal signal (S150), and when it is determined as the abnormal signal, this may be notified through an alarm or display (S151).

In this case, when it is determined as the abnormal signal because an ambient temperature or a temperature of the strip or blood is too much higher or lower than an indoor temperature, calculation of a blood glucose value using an artificial intelligence deep learning-based optimization algorithm may be performed without a waiting time.

In addition, when it is determined that it is not the abnormal signal, a blood glucose measurement value may be obtained from a response current measured by an artificial intelligence deep learning blood glucose measurement algorithm optimized by the artificial intelligence deep learning correction unit 17.

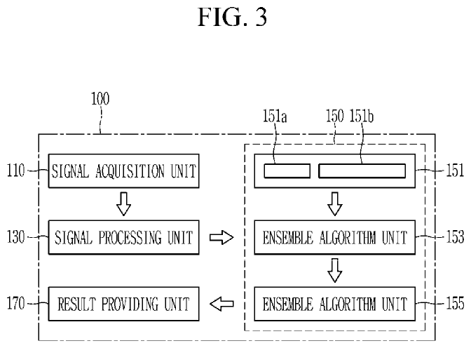

FIG. 3 illustrates a detailed view of an artificial intelligence learning calibrator in an internal configuration diagram of a blood glucose measurement device using artificial intelligence deep learning according to an exemplary embodiment of the present invention.

As illustrated in FIG. 3, the artificial intelligence learning correction unit 17 may download or store an artificial intelligence learning algorithm optimized through a wired/wireless network through software with respect to a biometric information analysis artificial intelligence-based deep learning server 100, and the biometric information analysis artificial intelligence-based deep learning server 100 may include: a signal acquisition unit 110 configured to acquire one-dimensional time series data through an electrochemical reaction that occurs by injecting blood collected through the biometric information measurement device 10 into a sensor (strip); a signal processing unit 130 configured to preprocess a signal to exclude abnormal signals due to blood injection abnormality and hardware abnormality among signals acquired from the signal acquisition unit 110 or to obtain an optimized biometric information measurement algorithm that is to be used in the biometric information measurement device 10; a biometric information measurement algorithm generating unit 150 configured to automatically extract features of the optimized biometric information measurement algorithm utilizing a deep learning artificial neural network technique by using the signal processed through the signal processing unit 130; and an optimization algorithm result providing unit 170 configured to be used in the biometric information measurement device 10.

The signal processing unit 130 may convert data into a certain size or distribution through normalization or standardization such that the biometric information measurement algorithm generation unit 150 can learn it, and the converted data may be converted into a data image by combining multi-channel data or using signal processing and data conversion such as domain conversion (e.g., time or frequency domain).

The biometric information measurement algorithm generator 150 may include: an algorithm structure unit 151 configured to form an algorithm structure for measuring blood glucose; an algorithm learning unit 153 configured to adjust variables in the algorithm to accurately predict a blood glucose value, where a predicted blood glucose value is true; and an ensemble algorithm unit 155 configured to calculate a final predicted value by combining one or more algorithms to improve accuracy and precision of blood glucose value prediction. In addition, the algorithm structure unit 151 may include: a feature extraction unit 151a configured to extract a feature of an analyte included in the biometric information signal data preprocessed by the signal processing unit 130; and a blood glucose value predicting unit 151b configured to estimate a blood glucose value by using features obtained by using the feature extracting unit 151a.

The algorithm structure unit 151 automatically extracts features reflecting the result value to be classified or measured from the image reflecting a surrounding environment, such as the components of the analyte, the hematocrit, the temperature, and the characteristics of the interfering species, utilizing a deep learning artificial neural network technique.

The algorithm learning unit 153 derives a weight and a bias between layers of artificial neural networks of which an error of a result value is minimal through an algorithm learning process using the extracted features.

The algorithm structure unit 151, which is an artificial neural network algorithm, may be utilized as a regression model to estimate a specific value depending on a purpose, and may also be used as a classifier to classify types of analytes.

In the present specification, correcting the occurrence of measurement errors due to hematocrit during blood glucose measurement is described as an exemplary embodiment, but the concentration of various metabolites, e.g., organic or inorganic substances such as beta hydroxybutyrate (aka ketone), cholesterol, lactate, creatinine, hydrogen peroxide, alcohol, amino acids, or glutamate may be corrected in the same way by introducing a specific enzyme like a glucose test. Accordingly, the present invention can be used to quantify various metabolites by varying types of enzymes included in sample layer composition.

For example, quantification of beta hydroxybutyrate, glucose, glutamate, cholesterol, lactate, ascorbic acid, alcohol, and bilirubin may be performed by using â-hydroxybutyrate dehydrogenase, glucose oxidase (GOx), glucose dehydrogenase (GDH), glutamate oxidase, glutamate dehydrogenase, cholesterol oxidase, cholesterol esterase, lactate oxidase, ascorbic acid oxidase, alcohol oxidase, alcohol dehydrogenase, bilirubin oxidase, etc. DeletedTextsâ

In the biometric information measurement device 10 according to an exemplary embodiment of the present invention, a working electrode and an auxiliary electrode are provided to face each other on different planes, and a face-to-face electrochemical biosensor coated with a reagent composition including an enzyme and an electron transport medium depending on a material may be applied to the working electrode.

In addition, in the biometric information measurement device 10 according to an exemplary embodiment of the present invention, a working electrode and an auxiliary electrode may be provided on one plane, and a planar electrochemical biosensor coated with a reagent composition including an enzyme and an electron transport medium depending on a material may be applied on the working electrode.

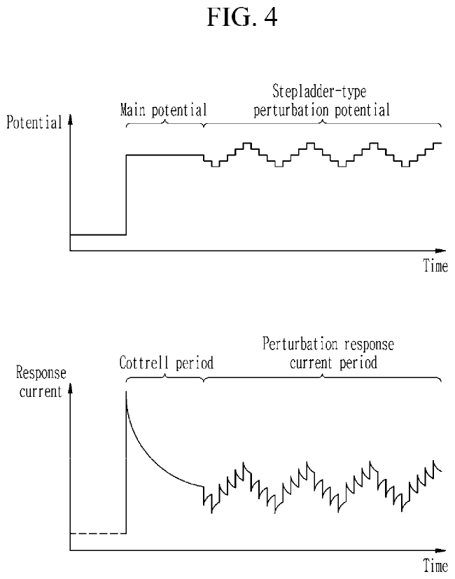

Now, a reason for using the Λ-stepladder-type perturbation potential will be described simply with reference to FIG. 4.

FIG. 4 illustrates a graph showing a Λ-stepladder-type perturbation potential used in a blood glucose measurement method using artificial intelligence deep learning and a corresponding sensitive current according to an exemplary embodiment of the present invention.

On the other hand, in accordance with the blood glucose measurement method using the artificial intelligence deep learning according to the exemplary embodiment of the present invention, as illustrated in FIG. 4, concentration of the bioanalyte is measured through the sensitive current by applying the stepladder-type perturbation potential after a constant voltage (VDC) is applied, and the application of the stepped ladder-shaped perturbation potential in this way causes an important change in the characteristics of the sensitive current, so as to eliminate or minimize an influence of an erythrocyte volume ratio or other interfering species.

Herein, the sensitive current is expressed as a first sensitive current or a second sensitive current to indicate that characteristics of the sensitive current are changed by fluctuation or perturbation and are different from each other.

A stepped ladder perturbation potential application method with periodicity that is additionally applied for a short period of time for the purpose of removing an effect of an erythrocyte volume ratio in a calibration formula after applying a constant voltage is referred to as “Λ-stepladder perturbation potential” or simply “stepladder potential.”

Currents with different characteristics refer to currents that can be used as a variable that can effectively separate or correct an effect of an erythrocyte volume ratio because a method depends on blood glucose and the erythrocyte volume ratio (blocking substance).

A method of finding feature points in the second sensitive currents corresponding to a period during which a perturbation potential is applied and a method of creating a feature from the feature points will be described as follows.

The method below is merely an example, and may be applied by various modifications depending on a purpose of application.

1) Sensitive currents near peak and valley voltages of a specific stepped ladder

2) Curvature of a curve consisting of the sensitive currents of each step in the stepped ladder

3) Difference between a current value at the stepped ladder peak and a current at the valley

4) Sensitive currents in the stepped ladder between uphill and downhill

5) Sensitive currents at a start point and an end point of each stepped ladder cycle

6) Average value of sensitive currents obtained from stepped ladder waves

7) Values that can be obtained by expressing the current values obtained from Features 1 to 6 as mathematical functions such as four arithmetic operations, exponents, logarithms, trigonometric functions, etc.

In this way, the feature points may be found in the second sensitive currents corresponding to the period during which the perturbation potential is applied, or the current values obtained from the feature points may be made into a feature, and these may be linearly combined to apply multivariable regression analysis, so that a calibration formula that minimizes the effect of the erythrocyte volume ratio may be obtained.

The calibration formula is one of

$$glucose = {\sum }_{j} {c}_{j} {f}_{j} ( i ) , \\ glucose = {\sum }_{j} {c}_{j} {f}_{j} ( i , T ) , and$$

$$ketone body = {\sum }_{j} {c}_{j} {f}_{j} ( i ) ,$$

Herein, i is a current value that is greater than or equal to one obtainable from the first and second sensitive currents, and T is an independently measured temperature value.

Referring back to FIG. 3, the signal acquisition unit 110 may acquire one-dimensional time series data through an electrochemical reaction that occurs by injecting drawn blood into a sensor strip.

The signal preprocessing unit 130 may image the one-dimensional time series data as two or more-dimensional data through signal processing and data conversion, and may use it as an input signal of an artificial neural network.

The biometric information measurement algorithm generation unit 150 automatically extracts features reflecting a result value to be classified or measured from an image reflecting the component of the analyte and the surrounding environment using a deep learning artificial neural network technique.

Algorithm learning is performed using the features extracted through the algorithm learning unit 153 to derive weights and biases between layers of artificial neural networks of which errors of a result value are minimal.

An artificial neural network algorithm may be utilized as a regression model to estimate a specific value depending on a purpose, and may also be used as a classifier to classify types of analytes.

In the analyte measurement method using artificial intelligence deep learning according to the exemplary embodiment of the present invention, experiments are conducted by reflecting various factors that affect blood glucose values in order to obtain the learning data to create an algorithm of a blood glucose meter.

The main factors affecting the blood glucose values include blood glucose, hematocrit, measured temperature, partial pressure of oxygen, and the like.

Blood samples of various experimental conditions are produced, and learning data for making an algorithm is obtained through repeated measurements.

The obtained learning data, which are one-dimensional time series data, represent an electrochemical reaction of the analyte over time.

Learning data is converted to a certain scale or distribution through normalization or standardization.

The converted data can be converted into a data image by combining multi-channel data or using signal processing and data conversion such as domain conversion (e.g., time or frequency domain).

The imaged data may learn an algorithm that can output an appropriate result depending on an input using artificial neural network deep learning technique.

The artificial neural network is a method that mimics a principle of an operation of a human brain, and it is desirable to control weights between neurons in several layers in the artificial neural network.

These artificial neural networks include convolutional neural networks (CNNs), deep belief networks (DBNs), and recurrent neural networks (RNNs) depending on their structure. A difference between the artificial neural network deep learning technique and other machine learning is that features can be automatically extracted.

There are several methods for automatic feature extraction, such as restricted Boltzmann machines (RBM).

RBM may be used for unsupervised learning, and may go through an optimization process that makes the distribution of the input data and the distribution of reconstructed data that is determined (stochastic decision) depending on probability similar.

As a result, a result value of a hidden layer may be used as a feature value representing the input data.

It is possible to construct the structure of the artificial neural network by expanding the hidden layer into several layers to obtain the characteristics of each hidden layer, by repeatedly performing the above process.

A purpose of artificial neural network learning is to minimize output errors.

Methods for minimizing output errors include Levenberg-Marquardt, Gauss-Newton, Gradient descent, and the like.

Through the above optimization method, weight and bias values of the entire artificial neural network are determined.

In addition to weights and biases in artificial neural networks, an activation function also plays an important role.

The activation function determines how to receive an input signal from each neuron (node) and to send an output.

The activation function includes sigmoid, hyperbolic tangent, rectified linear unit, and the like.

The artificial neural network may be used for a classifier that classifies a type of data through a change in the activation function or structure of the output layer, or for regression that estimates a value.

For example, it may be used as a classifier that separates human blood from a control solution, and may also be used for regression analysis to estimate blood glucose values.

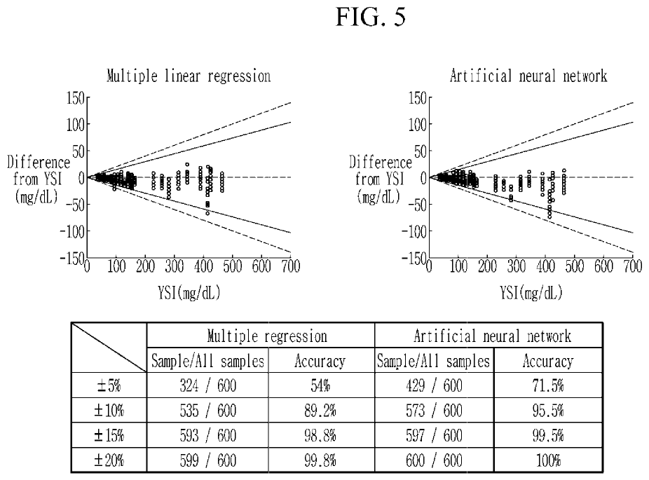

As illustrated in FIG. 5, results of a blood sugar estimation method using a conventional multiple regression method and a regression model using an artificial neural network to estimate blood glucose may be compared.

As a result of comparing an estimated blood glucose value with that of a YSI analyzer, which is a blood glucose reference measurement device, it can be seen that the estimated blood glucose value using an artificial neural network shows more accurate results than that of the conventional multiple regression method.

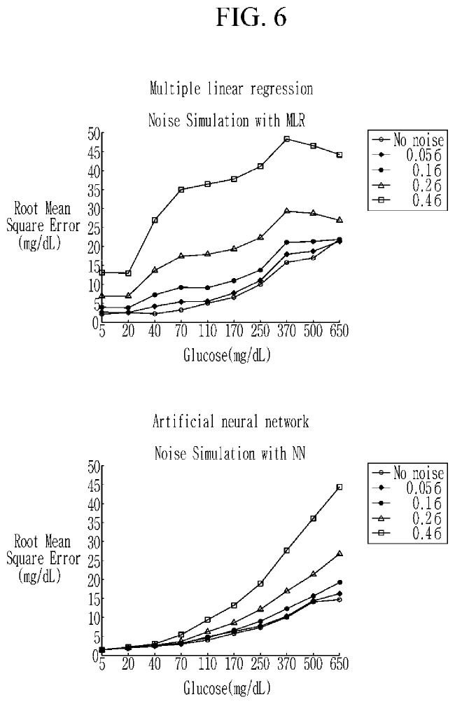

As illustrated in FIG. 6, when random noise of a predetermined magnitude is applied to an input signal, robustness of the algorithm depending on the noise may be confirmed.

Accordingly, the robustness of the algorithm was confirmed by a root mean square error of the YSI value, which is the reference value, and a measurement algorithm value.

In the case of multiple regression, it reacts sensitively to noise and the output value appears unstable regardless of blood concentration.

On the other hand, in the case of the artificial neural network, particularly at low concentration, it can be seen that it is robust against noise and the output value is more stable than with the regression method.

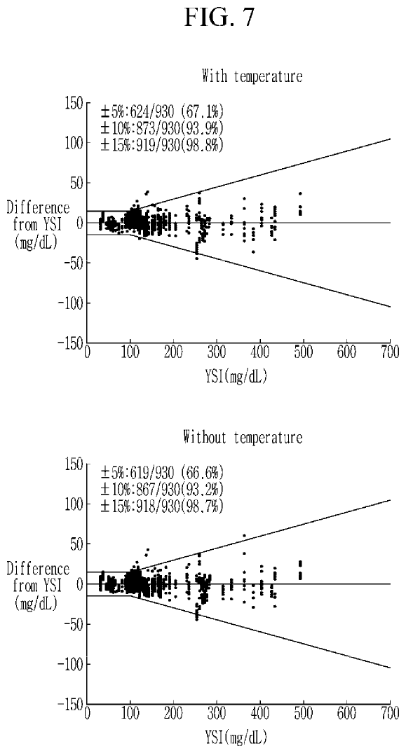

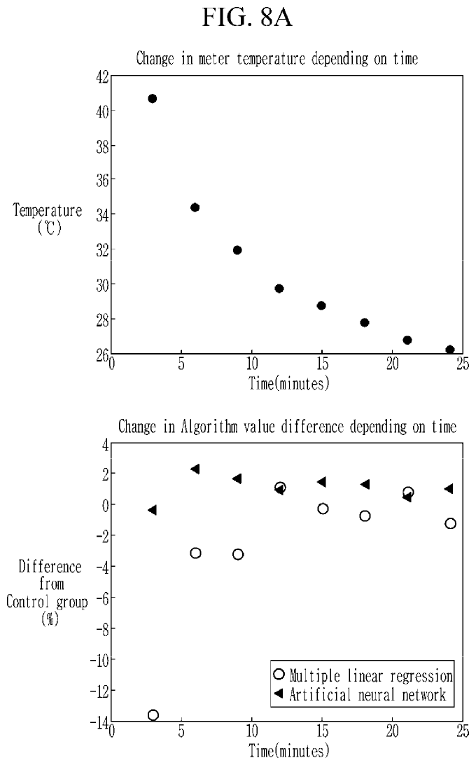

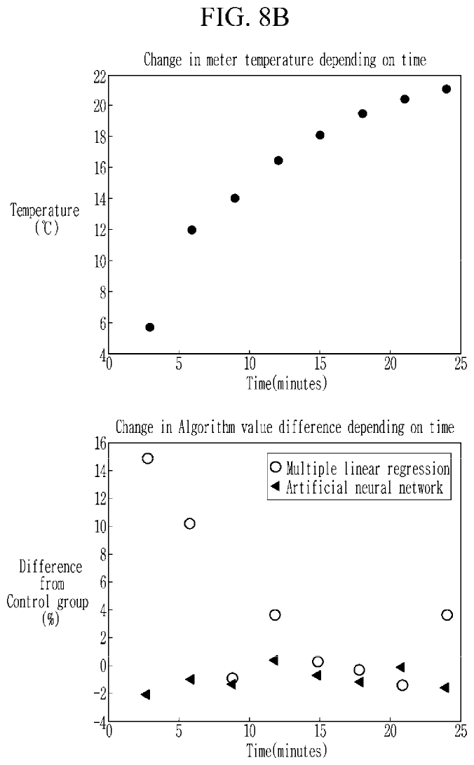

When a difference between the ambient temperature and the balancing temperature, which is one of the factors that can affect blood concentration, is large, an analyte concentration measurement method using artificial intelligence deep learning according to an exemplary embodiment of the present invention will be described by way of examples with reference to FIG. 7 to FIG. 8B.

FIG. 7 illustrates an exemplary diagram showing results of a regression model in which blood glucose estimation is performed when a temperature feature is not included and when the temperature feature is included depending on an analyte concentration measurement method utilizing artificial intelligence deep learning according to an exemplary embodiment of the present invention, and FIG. 8A and FIG. 8B respectively illustrate graphs showing robustness of an algorithm when an ambient temperature is higher than room temperature and lower than room temperature depending on an analyte concentration measurement method utilizing artificial intelligence deep learning according to an exemplary embodiment of the present invention.

### (Example 1) Performance Comparison Depending on Presence or Absence of a Temperature Feature of a Neural Net Model

Two identical artificial intelligence deep learning algorithm models including/without a temperature feature in the neural net algorithm of a same structure were implemented and shown in Table 1 and FIG. 7 in order to observe a performance change in a neural net algorithm depending on presence or absence of the temperature feature.

Performance comparison was confirmed through a total of 930 samples and clinical tests of a total of 9 lots.

As illustrated in FIG. 7, when a difference between YSI values, which are reference values, in the cases of including and not including the temperature feature, a case of ±5% shows average performance accuracy of 66.6% and 67.1%, a case of ±10% shows average performance accuracy of 93.2% and 93.9%, and a case of ±15% shows average performance accuracy of 98.7% and 98.8%, so it was confirmed that there was little difference in the performance of the neural net algorithm depending on presence or absence of the temperature feature in the clinical data.

TABLE 1

| | Artificial neural network | | | Artificial neural network | | |
|---|---|---|---|---|---|---|
| | without temperature | | | including temperature | | |
| Lot | ±5% | ±10% | ±15% | ±5% | ±10% | ±15% |
| 1 | 70.8 | 92.5 | 100 | 67.9 | 92.5 | 99.1 |
| 2 | 72.6 | 98.1 | 100 | 69.8 | 98.1 | 99.1 |
| 3 | 60.4 | 92.5 | 100 | 58.5 | 93.4 | 100 |
| 4 | 66.7 | 92.2 | 98 | 68.6 | 96.1 | 99 |
| 5 | 61.8 | 92.2 | 97.1 | 64.7 | 90.2 | 97.1 |
| 6 | 69.6 | 96.1 | 100 | 71.6 | 98 | 100 |
| 7 | 63.7 | 92.2 | 99 | 72.5 | 96.1 | 100 |
| 8 | 73.5 | 93.1 | 98 | 71.6 | 93.1 | 99 |
| 9 | 55.9 | 88.2 | 96.1 | 58.8 | 87.3 | 98 |
| Total | 66.6 | 93.2 | 98.7 | 67.1 | 93.9 | 98.8 |

### (Example 2) Comparison of Changes in Algorithm Results for Meter Temperature Differences According to Rapid Environmental Changes

A value of the temperature feature applied to the algorithm is a value obtained through a temperature sensor attached to a meter, and when there is a sudden change in the surrounding environment, it is difficult to measure the correct temperature immediately and time is required to achieve temperature equilibrium.

Therefore, when in a rapid environment change due to these problems, e.g., when entering a warm house from outside on a cold winter day, in order to use the meter, it is required to wait a certain time for temperature equilibration, otherwise, an incorrect blood glucose value may be obtained.

In contrast, with reference to Experimental Example 1, it will be described that such a problem can be solved by a bioanalyte concentration measurement method utilizing artificial intelligence deep learning according to an exemplary embodiment of the present invention, which does not include temperature.

For this purpose, in a process of equilibrating the temperature from 43 to 23° C. and a process of equilibrating the temperature from 0 to 23° C. with a control group experimenting in a 23° C. environment after equilibrating the temperature at 23° C., a difference in the algorithm values of experimental groups 1 and 2 according to the error of the measured temperature of the meter was compared with the case of the conventional multiple regression analysis method and the neural net algorithm without temperature was shown in Table 2 and FIG. 8A and FIG. 8B.

TABLE 2

| | | Multiple linear regression | | | Artificial neural network | | |
|---|---|---|---|---|---|---|---|
| | | | | Bias from | | | Bias from |
| Time | Temperature | Control | Experimental | control | Control | Experimental | control |
| (minutes) | (°C.) | group | group 1 | group (%) | group | group 1 | group (%) |
| 3 | 40.7 | 126 | 110 | −13.6 | 133 | 132 | −0.4 |
| 6 | 34.4 | 125 | 121 | −3.2 | 129 | 132 | 2.2 |
| 9 | 32.0 | 123 | 119 | −3.3 | 131 | 133 | 1.6 |
| 12 | 29.8 | 122 | 123 | 1.0 | 130 | 132 | 0.9 |
| 15 | 28.8 | 122 | 121 | −0.3 | 129 | 131 | 1.4 |
| 18 | 27.8 | 125 | 124 | −0.8 | 129 | 130 | 1.2 |
| 21 | 26.8 | 120 | 121 | 0.7 | 129 | 129 | 0.4 |
| 24 | 26.2 | 122 | 120 | −1.3 | 129 | 130 | 1.0 |
| | | | | Bias from | | | Bias from |
| Time | Temperature | Control | Experimental | control | Control | Experimental | control |
| (minutes) | (°C.) | group | group 2 | group (%) | group | group 2 | group (%) |
| 3 | 5.8 | 131 | 152 | 14.8 | 145 | 142 | −2.1 |
| 6 | 12.0 | 127 | 141 | 10.1 | 143 | 141 | −1.0 |
| 9 | 14.1 | 133 | 132 | −0.9 | 142 | 140 | −1.4 |
| 12 | 16.4 | 132 | 136 | 3.6 | 141 | 142 | 0.4 |
| 15 | 18.1 | 132 | 132 | 0.3 | 142 | 141 | −0.7 |
| 18 | 19.4 | 129 | 129 | −0.3 | 139 | 138 | −1.1 |
| 21 | 20.4 | 132 | 130 | −1.4 | 138 | 138 | −0.1 |
| 24 | 21.1 | 131 | 135 | 3.5 | 141 | 139 | −1.6 |

As an experiment result, 1 meter of the experimental group in the 43° C. environment shows a difference in the experimental environment of 23° C. and the measured temperature value until about 24 minutes pass. This difference with the measured temperature shows a maximum difference of 13.6% from the control group in the multiple linear regression algorithm, and a waiting time for temperature equilibration of about 9 minutes was required. On the other hand, the artificial neural network algorithm maintained a difference of 0 to 2% from that of the control group regardless of the difference in temperature, and was immediately available without a waiting time.

Similarly, 2 meter of the experimental group in the 58° C. environment shows a difference in the experimental environment of 23° C. and the measured temperature value until about 24 minutes pass. This difference with the measured temperature shows a maximum difference of 14.8% from the control group in the multiple linear regression algorithm, and a waiting time for temperature equilibration of about 15 minutes was required.

On the other hand, the artificial neural network algorithm maintained a difference of 0 to 2% from that of the control group regardless of the difference in temperature, and was immediately available without a waiting time.

Through the experimental result, it was confirmed that the neural net algorithm without temperature can obtain more accurate results for rapid environmental changes, and that users can use the product without a special waiting time.

1. A bioanalyte concentration measurement method using artificial intelligence deep learning, the method comprising:
injecting a liquid biological sample into a sample cell having a working electrode and an auxiliary electrode, in which an electron transport medium and a redox enzyme which is capable of catalyzing a redox reaction of an analyte are fixed;
obtaining a first sensitive current at a characteristic point of at least one point of time by applying a constant DC voltage to the working electrode to initiate the redox reaction of the analyte and to proceed with an electron transfer reaction;
obtaining a second sensitive current at two or more points of time by applying a Λ-stepladder-type perturbation potential after applying the constant DC voltage;
calculating a predetermined feature from the first sensitive current or the second sensitive current; and
correcting concentration of the analyte by using a calibration formula composed of at least one feature function by artificial intelligence learning such that an influence of at least one interfering substance in the biological sample is minimized,
wherein the correcting of the concentration of the analyte includes calculating a new feature by reacquiring the first and second sensitive currents by artificial intelligence learning, and
the artificial intelligence learning includes: obtaining blood samples of various experimental conditions that are produced, and learning data for making an algorithm through repeated measurements;
the obtained learning data, which is one-dimensional time series data, representing an electrochemical reaction of the analyte over time;
converting data is converted to a certain scale or distribution through normalization or standardization of the learning data;
the converted data being signal-processed by combining multi-channel data or performing domain conversion; and
learning an algorithm that can output an appropriate result depending on an input using artificial neural network deep learning technique.

2. The bioanalyte concentration measurement method of claim 1,
which is a method of making a feature with a feature point by selecting a feature point having a different linear dependence on the analyte and an interfering material in the first or second sensitive currents, constructing a feature from the feature point, creating a test formula composed of the above feature by the artificial intelligence learning uses the second sensitive current near a peak and valley voltage of a specific stepped ladder, curvature of the curve consisting of the sensitive currents of each step in a stepladder perturbation potential, a difference between the current value at the peak and the current value at the valley of the stepladder perturbation potential, sensitive currents at the stepladder perturbation potential between uphill and downhill, sensitive currents at a start point and an end point of the cycle of each stepladder perturbation potential, one of the average values of the sensitive currents obtained from the stepladder perturbation potential, or values that can be obtained by expressing the current values obtained from this with mathematical functions such as four arithmetic operations, exponential, logarithmic, trigonometric functions, etc.

3. The bioanalyte concentration measurement method of claim 1, wherein
the second sensitive current is obtained within 0.1 to 1 s after obtaining the first sensitive current.

4. The bioanalyte concentration measurement method of claim 1, wherein
the artificial intelligence learning corrects at least two interfering species among a concentration abnormality of the analyte in the biological sample, contamination of the analyte in the biological sample, incorrect use of the strip containing the analyte in the biological sample, an ambient temperature, an electrode material, an electrode arrangement method, a flow path shape, characteristics of reagents used, and abnormality in the concentration measuring device of the analyte in the biological sample.

5. The bioanalyte concentration measurement method of claim 1, wherein
even when an ambient temperature change is large, concentration measurement of the analyte in the biological sample is corrected by artificial intelligence deep learning without a waiting time for temperature balancing.

6. The bioanalyte concentration measurement method of claim 1, wherein
the calibration formula is one of
$$glucose = {\sum }_{j} {c}_{j} {f}_{j} ( i ) , \\ glucose = {\sum }_{j} {c}_{j} {f}_{j} ( i , T ) , and$$
$$ketone body = {\sum }_{j} {c}_{j} {f}_{j} ( i ) ,$$
wherein i is a current value that is greater than or equal to one obtainable from the first and second sensitive currents, and T is an independently measured temperature value.

7. The bioanalyte concentration measurement method of claim 1, further comprising:
adjusting weights between neurons present in several layers in the artificial neural network; and the artificial neural network automatically extracting features using one of convolutional neural networks (CNN), deep belief networks (DBN), and recurrent neural networks (RNNs) according to a structure.

8. The bioanalyte concentration measurement method of claim 1, wherein
the feature automatic extraction uses restricted Boltzmann machines (RBM), and includes optimizing in which distribution of the input data and distribution of reconstructed data determined (stochastic decision) according to a probability are similar.

9. The bioanalyte concentration measurement method of claim 8, wherein
the optimizing includes determining a weight and a bias value of the entire artificial neural network, and using an activation function.

10. The bioanalyte concentration measurement method of claim 9, wherein
the artificial neural network is used for a classifier that classifies a type of data through a change in the activation function or structure of the output layer, or for regression that estimates a value.
